(19) United States
(12) Patent Application Publication
(10) Pub. No.: US 2008/0090289 A1
(43) Pub. Date: Apr. 17, 2008

(54) Microbial Sampling Device

(75) Inventors: Adrian J. Denvir, Richardson, TX (US); Rattaya Chow Yalamanchili, Houston, TX (US); Marius Raducanu, College Station, TX (US); Mark Flusche, College Station, TX (US); Mitchell A. Anderson, Stafford, TX (US)

Correspondence Address: STREETS & STEELE, 13831 NORTHWEST FREEWAY, SUITE 355, HOUSTON, TX, 77040, US

(73) Assignee: LYNNTECH, INC., College Station, TX (US)

(21) Appl. No.: 11/758,506

(22) Filed: Jun. 5, 2007

Related U.S. Application Data
(60) Provisional application No. 60/804,071, filed on Jun. 6, 2006.

(51) Int. Cl. C12M 1/26

(52) U.S. Cl. 435/309.1

(57) ABSTRACT

A method and apparatus for sampling microorganisms using fluid pressure, vacuum or a combination thereof to create fluid flow that collects biological and chemical material on a filter element. The filter element is secured in a cartridge replaceably coupled to a hand-held sample collection device that provides a fluid drain downstream of the filter element. The cartridge allows a fluid to pass through the filter element in a manner that prevents cross-contamination of subsequent samples. The sample makes contact with only the filter cartridge before the fluid is drained. Accordingly, the source of the fluid is not contaminated because it remains at a higher pressure than either the sample or the fluid drain during operation. Subsequent filter elements avoid contamination by biological or chemical material from a previous sample.

This application claims priority from U.S. provisional application 60/804,071 filed on Jun. 6, 2006.

### FIELD OF THE INVENTION

The present invention relates to an apparatus and method for automated sampling of microorganisms onto a microbial filter.

### BACKGROUND OF THE RELATED ART

Wet swabbing is presently the most widely used method for collecting samples of biological or chemical materials from a surface for analysis. This method involves drawing a swab across a surface area in a specific manner, such as with the swab at a certain angle relative to the surface while being axially rotated so that the sample is lifted away from the surface. This method requires training, experience and extreme diligence to perform. Even still, the results can be inconsistent.

Bradley (U.S. Pat. No. 5,868,928) has proposed a microbial sampling, filtration, and recovery device requiring multiple chambers assembled in two configurations. A first configuration for collecting a microbial sample includes a surface nozzle, a prefilter chamber, a microbial filter chamber and a vacuum source connected in this order. The device utilizes a wash solution that is applied to a surface, such as a meat carcass, to suspend microbes. A vacuum source draws the microbe-containing solution and air through the nozzle and past certain prefilters before directing the solution into a chamber containing a microbial filter and a hydrophobic filter. The microbial filter is used to collect the suspended microbes from the wash solution and the hydrophobic filter allows air to exit the chamber to the vacuum source.

A second configuration of the device for recovering and concentrating the microbial sample includes the filter chamber connected between a rinse chamber and a recovery chamber. Accordingly, recovery of the microbial sample involves removing the filter chamber from the first configuration and assembling it into the second configuration. The microbial sample is then recovered by back flushing a rinse solution through the filter to dislodge and transport the microbes into a collection receptacle. Finally, the collection receptacle is centrifuged to concentrate the sample for use by some analytical means.

However, this device exposes a large number of parts to contamination. Before using the device to collect subsequent microbial samples, many of the parts must be thoroughly sterilized or discarded and replaced. This makes the method very costly and time consuming.

Therefore, there remains a need for an improved apparatus and method for collecting microbial or chemical samples without cross contamination. It would be desirable if the apparatus and method did not require sterilization or replacement of multiple components. It would also be desirable if the apparatus and method enabled collecting subsequent samples without elaborate reconfigurations of the device.

### SUMMARY OF THE INVENTION

The invention provides an apparatus for sampling microorganisms. Preferably the apparatus comprises a hand-held sampling device having a proximal end adapted for coupling in fluid communication with a fluid drain, a distal end including a coupling, and a first fluid passageway extending from the proximal end to the distal end. The apparatus preferably also comprises a replaceable sample collection cartridge having a proximal end adapted for selectively sealingly securing to the sampling device by engagement with the coupling, a second fluid passageway providing fluid communication between the first fluid passageway and a distal end of the cartridge, and a microorganism filter element extending across the second passageway and disposed a spaced distance from the distal end. A sample collection manifold is defined between the microorganism filter elements and the surface to be sampled, wherein the sample collection manifold provides fluid communication between a fluid source, the surface to be sampled, and the microorganism filter element. Furthermore, the apparatus provides sufficient differential pressure between the fluid source and the fluid drain to pass fluid from the fluid source across the surface to be sampled and through the microorganism filter element to the fluid drain such that a sample of microorganisms is collected from the surface to be sampled onto the microorganism filter element. Optionally, the fluid source is a gas selected from an inert gas, air, carbon dioxide, and combinations thereof. Alternatively, the fluid may be a liquid, such as an aqueous fluid.

In one embodiment, the sample collection cartridge can be disengaged from the coupling after collecting the sample and a subsequent sample collection cartridge can be engaged with the coupling for collection of a subsequent sample that is free of cross-contamination from any previous sample. The coupling may include, for example, a latch-in-place mechanism, a friction fitting, snap-on elements, and combinations thereof. The sample collection cartridge is preferably an aseptic disposable single-use cartridge stored in an aseptic container prior to coupling with the sampling device, and wherein the microorganism filter element does not come into contact with any other surface or fluid that has been cross contaminated. Preferably, the microorganism filter element is spaced less than 3 centimeters from the distal end of the sample collection cartridge.

In a further option, the sample collection cartridge is aseptically dispensable from the sampling device into a container of recovery solution. A suitable latch-in-place mechanism includes a quick release trigger for removing the sample collection cartridge from the sampling device. The sample collection cartridge is operationally independent of the orientation of the surface to be sampled. Optionally, the handheld device can sample surfaces, liquids and gases.

In a further embodiment, the apparatus comprises a rinse reagent supply channel extending through the sample collection cartridge and terminating at a recessed face. The rinse reagent supply channel provides fluid communication with the sample collection manifold adjacent the perimeter of the manifold for delivering a rinse reagent across the surface to the sampled to the microorganism filter element. Preferably, the depth of the recessed face provides turbulent fluid communication with a known reagent source. The recessed surface may be bounded by a sealing member that forms a temporary seal against the surface to be sampled. The rinse reagent is selected from liquids, gases, and combinations thereof.

In yet another embodiment, the sampling cartridge comes in various sizes to accommodate access to crevices. Accordingly, the apparatus may be provided with two or more sample collection cartridges with distal ends having different shapes for collecting samples from different surface geometries or locations.

### BRIEF DESCRIPTION OF THE DRAWINGS

FIG. 1 is a schematic cross-sectional view of an apparatus for sampling microorganisms.

FIGS. 2A and 2B are schematic fluid flow diagrams illustrating how a differential pressure is created to impart flow to the motive fluid.

FIG. 3A is a flow diagram of an apparatus for collecting samples.

FIG. 3B is a cross-sectional side view of the sampling device of FIG. 3A shown in greater detail.

FIGS. 4A and 4B include an exploded view and an assembled perspective view of a disposable filter cartridge.

FIG. 5 is a perspective view of a self-contained sample collection apparatus in the form of a wand.

FIG. 6 is a bar chart showing the relative amounts of RNA detected from samples collected by a standard wet swab technique and using a vacuum sampling device of the invention.

### DETAILED DESCRIPTION

The present invention provides a method and apparatus for sampling microorganisms using fluid pressure, vacuum or a combination thereof in order to create a differential pressure and fluid flow that collects biological and chemical material on a filter element. The filter element is secured in a cartridge that can be replaceably coupled to a hand-held sample collection device that provides a fluid drain, or fluid communication with a fluid drain, downstream of the filter element. The cartridge is designed to allow a fluid to be passed through the filter element in a manner that prevents cross-contamination of subsequent samples collected with the hand-held sample collection device. The sample being collection makes contact with only the filter cartridge before the fluid is removed to the drain in the sample collection device. Accordingly, the source of the fluid is not contaminated because remains at a higher pressure than either the sample or the fluid drain during operation. The cartridge, and particularly the filter element of the cartridge, will become contaminated, but the filter element collects the biological and/or chemical material for analysis and the cartridge is replaced between samples. Preferably, the cartridge disposable before or after analysis of the sample collected on the filter element. While the fluid drain could potentially become contaminated by biological or chemical material that passes through the pores of the filter element, such contamination is likely to be minor and, most importantly, is downstream of the position in which replacement filter cartridges are coupled. Accordingly, subsequent filter elements do not become contaminated by biological or chemical material from a previous sample.

In one embodiment, the hand-held sampling device has a proximal end adapted for coupling in fluid communication with a fluid drain, a distal end including a coupling, and a first fluid passageway extending from the proximal end to the distal end. The hand-held sampling device may be a sampling head that provides physical and fluidic connections between the filter cartridge and tubing that leads to a remote drain, such as a vacuum chamber or generator. Alternatively, the hand-held sampling device may be directly coupled to, or contain within it, the fluid drain.

While the fluid source may be any gas or liquid that will not interfere with the analysis of the biological or chemical sample or damage and a valuable substrate from which the sample is being taken, the invention lends itself to a wide variety of fluids, including gases, liquids, combinations of gases, combinations of liquids, or combinations of one or more gases with one or more liquids. The selection of fluids may be handled on a case-by-case basis given due consideration for the analytical technique to be performed on the sample, the nature of the substrate, and the condition or type of biological or chemical species being sampled. For example, if the species is believed to be easily collected, then ambient air may be the preferred fluid since it is available from the surrounding environment. However, if the air from the surrounding environment may itself be contaminated, the preferred fluid may be an inert gas, such as nitrogen, argon, carbon dioxide, or combinations thereof. Still further, if the sample is water-borne or can be loosened or drawn out of a porous substrate with water, then water may serve as the fluid. Even on a solid surface, the fluid may be water or an aqueous solution.

In mixed fluid systems, it may also be useful to consider that one fluid may be the primary motive fluid, while another fluid serves a collection aid. Some embodiments of the invention may utilize air or an inert gas as the standard primary motive fluid, but include integral or separate facilities for delivery of a different fluid as a collection aid. A collection aid is selected to increase the collection of the biological or chemical material from the substrate, through one or more phenomena, such as dissolution of the material into the collection aid. Other collection aids may include, for example, surfactants or buffers. One mixed fluid system includes the initial application of a collection aid, such as water, onto a surface to be sampled, such as a wood desktop, followed by positioning of the sampling apparatus with the filter cartridge disposed against the surface and drawing air or an inert gas across the wet surface and through the filter element. Still, the collection aid may be applied before and/or during the sample collection using the motive fluid.

Where the fluid source is ambient air, the drain will typically release the air back into the surrounding environment. Inert gases may be disposed of similarly in small quantities. When the drain receives both gases and liquids, it is preferred that the drain system include a gas/liquid separator to facilitate separate handling or disposal of these fluids. In portable systems, gases such as air will be immediately released into the surrounding environment to reduce the required storage capacity of the drain system. Liquids will typically be collected in a reservoir for batch-wise disposal, although they could likewise be continuously released or disposed of.

The combination of fluid drain and fluid source must provide a pressure differential that will cause sufficient fluid flow from the source to the drain in order to collect the sample. The pressure differential may be caused by any suitable combination of source pressure/drain pressure, such as atmospheric source pressure/vacuum drain pressure, high source pressure/atmospheric drain pressure, or high source pressure/vacuum drain pressure. The exact pressure differential may vary significantly depending upon the nature and condition of the sample to be collected. However, a pressure differential of between about 2 psig and about 14 psig is preferred. In one preferred embodiment, the dimensions of the filter cartridge are taken into consideration along with the differential pressure of the motive fluid in order to provide turbulent fluid flow conditions across the surface to be sampled. The fluid turbulence is believed to enhance the sample collection because of the physical forces imparted on the biological or chemical material. It is also preferred to distribute the motive fluid generally about the perimeter of the manifold or filter element, in order to collect the sample from the entire surface within that perimeter. Supplying the motive fluid to a single point may allow channeling of the motive fluid such that only a fraction of the surface is exposed to conditions appropriate to collect a sample

The replaceable sample collection cartridge has a proximal end adapted for selectively sealingly securing to the sampling device by engagement with the coupling. Such a seal may be accomplished in a variety of ways, such as securing a gasket or seal ring to either the proximal end of the cartridge or the distal end of the sampling device. Alternatively, close fitting of the filter cartridge to the sampling device may be sufficient to maintain the necessary differential pressure and/or prevent spread of contaminants out of the system. This is particularly suitable in a sampling device that uses vacuum drain pressure and will not be contaminated merely by drawing in small amounts of air.

The sampling device and the replaceable sample collection cartridge, alternatively referred to as the filter cartridge, are selectively secured or coupled together during collection of a sample. The means by which the two components are secured is very wide ranging and includes almost any type of nonpermanent fastener that leaves the sampling device in good condition to receive subsequent cartridges. Examples of possible fastener mechanisms include adhesives, magnets, hook-and-loop fasteners, screw threads, biasing latches, snap-on elements and frictional fittings or engagement. Typically, the connection between the two components will not undergo any high stresses or loads, but should retain the cartridge in an operable position with respect to the sampling device. Still, accidental release of the cartridge is preferably avoided since it could jeopardize the integrity of the sample being collected. Most preferably, the coupling that secures the cartridges is a quick-release mechanism. The release trigger may be located on either the sampling device or the filter cartridge, but is preferably located on the sampling device.

A fluid passageway through the cartridge provides fluid communication between the fluid passageway in the sampling device and a distal end of the cartridge. The microorganism filter element is secured in the cartridge and extends across the fluid passageway within the cartridge. The filter element may be permanently or temporarily secured in place, and preferably receives physical support on the downstream side of the filter element (the proximal end) by a macroporous support, such as a plastic or metal mesh, screen or grid. Preferably, the filter element extends entirely across the fluid passageway so that no fluid can reach the fluid drain without passing through the pores of the filter element. Furthermore, the filter element is preferably disposed a spaced distance from the distal end that does not exceed one inch, and more preferably less than 0.5 inch.

The space between the microorganism filter element and the surface to be sampled defines a sample collection manifold. Preferably, the microorganism filter element is spaced less than 3 centimeters from the distal end of the sample collection cartridge. When the sample collection manifold is disposed against a surface, the manifold provides fluid communication between a fluid source, the surface to be sampled or other source to be sampled, and the microorganism filter element. In operation, applying sufficient differential pressure between the fluid source and the fluid drain causes fluid to pass from the fluid source across the surface to be sampled and through the microorganism filter element to the fluid drain such that a sample of microorganisms is collected from the surface to be sampled and deposited onto the microorganism filter element. Because the filter element is disposed is close proximity to the surface to be sampled, the only components of the device that become contaminated with the sample are either part of the sample collection cartridge or are downstream of the filter element. The used sample collection cartridge is removed and replace with a new cartridge before taking another sample, such that the cartridge is not a source of cross contamination. The sampling device, while potentially contaminated, is downstream of the filter element of the new cartridge and contaminants contained therein, such as in the first fluid passageway, will be drawn further away from the filter element. Accordingly, replacing the cartridge between samples prevents any cross contamination of samples. Replacement of the cartridges is facilitated by the cartridges placement at the extreme distal end of the sampling device. Most preferably, the distal end of the cartridge forms the sampling nozzle that makes contact with the surface to be sampled. Optionally, this distal end may form a seal against the surface, especially if an inert motive fluid is to be used in order to avoid ambient contamination of the sample. Still, the distal end may be configured in a manner that allows ambient air to be drawn into the manifold region as the motive fluid.

The operation described above does not depend upon a specific orientation of the filter element or the surface to be sampled with respect to gravity. Specifically, the apparatus operates at the same efficiency, accuracy and operability regardless of physical orientation to collect samples from floors, walls, ceilings and the like. Consequently, the sample collection cartridge is operationally independent of the orientation of the surface to be sampled. Furthermore, the sampling cartridge may be provided in various sizes to accommodate access to crevices or other difficult geometries. Preferably, the apparatus is used with a kit of two or more sample collection cartridges with distal ends having different shapes for collecting samples from different surface geometries or locations. Still further, the sample may be collected from a gas or liquid, such as ambient air or a body of water.

Whereas the fluid drain may optionally collect one or more of the fluids for batch-wise disposal or analysis, such is not a necessary aspect of the invention. In fact, the fluids at the drain would likely contain cross contamination from each of the samples taken since the entire system was new or last cleaned. Rather, it is intended that only the biological or chemical sample collected on the filter element will be used for analysis.

It is preferred to take precautions to store the filter cartridges in an aseptic container prior to use. A preferred aseptic container is a sealed plastic pouch containing a single filter cartridge, wherein the pouch can be opened immediately before use. The same or different container may then be used to store the used filter cartridge awaiting analysis. In a particularly preferred embodiment, the sampling device can secure and withdraw the cartridge from an aseptic container without requiring human contact with the cartridge. Similarly, the sampling device may include a quick-release mechanism that allows the cartridge to be released directly into an aseptic container following sample collection. Most preferably, the filter cartridge is a single-use, disposable cartridge

An optional embodiment of the invention further includes a rinse reagent supply channel extending through the sample collection cartridge and terminating in fluid communication with the sample collection manifold. A rinse reagent is a fluid, typically a liquid or gel, but is potentially a gas such as ozone, that is applied to a surface in order to improve the collection of biological or chemical materials from the surface. Accordingly, the rinse reagent may be directly applied to the surface using the same device that will be used to collect the sample. While rinse reagents may also be applied separately, such as with a stand-alone spray bottle, this embodiment provides rinse reagent in a controllable manner into the manifold area. The rinse reagent may be applied before and/or during collection of the sample with a motive fluid. Furthermore, the rinse reagent may improve collection through any mechanism, such as physical, chemical or otherwise. Preferably, the rinse reagent channel distributes the rinse reagent over a major portion of the surface that is being sampled, i.e., the surface exposed to the sample collection manifold. Preferably, the apparatus does not damage the surface being sampled, either through physical and chemical actions.

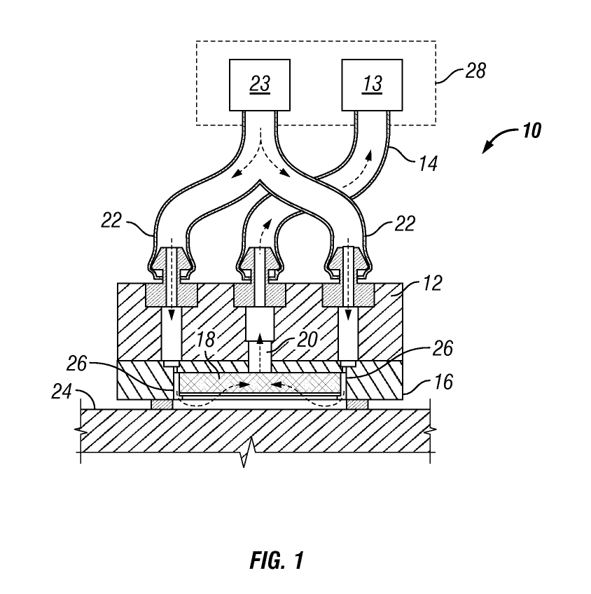

FIG. 1 is a schematic cross-sectional view of an apparatus 10 for sampling microorganisms. The apparatus includes a sampling device 12 that is in fluid communication with a fluid drain 13 through the tubing 14. A cartridge 16 is replaceably secured to the sampling device 12 while taking a sample from a surface 24 to be sampled. The cartridge securely holds a filter element 18 that is disposed across a passageway 20 that leads to the tubing 14 and ultimately to the fluid drain 13. Although a fluid source may simply pass through or around the cartridge 16, this embodiment provides fluid communication of a fluid from a fluid source 23 via supply tubing 22 through both the sampling device 12 and the cartridge 16. The fluid is delivered over a surface 24 to be sampled, preferably through a channel 26 forming a perimeter around an area where a sample is to be collected. In operation, the motive fluid flows from the fluid source 23 to the fluid drain 13 because of a pressure differential. As shown, the fluid source 23 and the fluid drain 13 are disposed in a common housing 28 that, depending upon the length of the supply tubing 22 and the drain tubing 14, may facilitate the sampling device 12 and cartridge 16 being independently maneuverable and preferably hand-held. Alternatively, the sampling device 12 and the fluid source and/or fluid drain may be an integral unit.

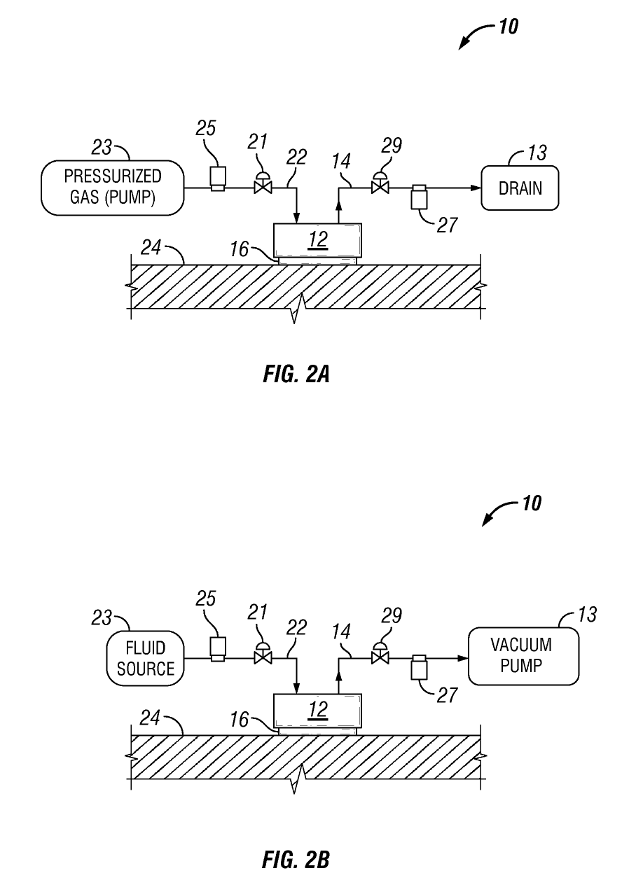

FIGS. 2A and 2B provide schematic fluid flow diagrams illustrating how a differential pressure may be created to impart flow to the motive fluid. In FIG. 2A, the fluid source 23 includes a fluid, such as a gas or liquid that is pressurized, either from a pump, compressor or pressure vessel, to a pressure that is greater than the pressure of the fluid drain 13. The apparatus may optionally include an accumulator 25 and/or a liquid trap 27. In operation, a fluid supply valve 21 and any fluid drain valve 29 are opened to initiate fluid flow through tubing 22, across surface 24, into tubing 14, to the drain 13. Alternatively, in FIG. 2B, the drain 13 includes a vacuum source, such as a pump, so that upon opening of the valves, fluid flows through the same passageways. The primary difference between the Figures is that the tubing, sampling device and cartridge will be at positive pressure (above atmospheric) in FIG. 2A and negative pressure (below atmospheric) in FIG. 2B. The different pressure regimes may affect specific design elements of a final product as will be apparent to one skilled in mechanical design. Furthermore, the selection of positive or negative pressure may be influenced by the location and nature of the sample being taken. For example, a positive pressure system could leak contaminants collected from a surface, whereas a negative pressure system could draw in contaminated ambient air. Further still, a third embodiment can be envisioned that combines the use of a pressurized fluid source 23 with a vacuum fluid drain 13. This combination may be particularly useful when using an inert fluid, such as carbon dioxide gas, to avoid drawing in excessive amounts of ambient air.

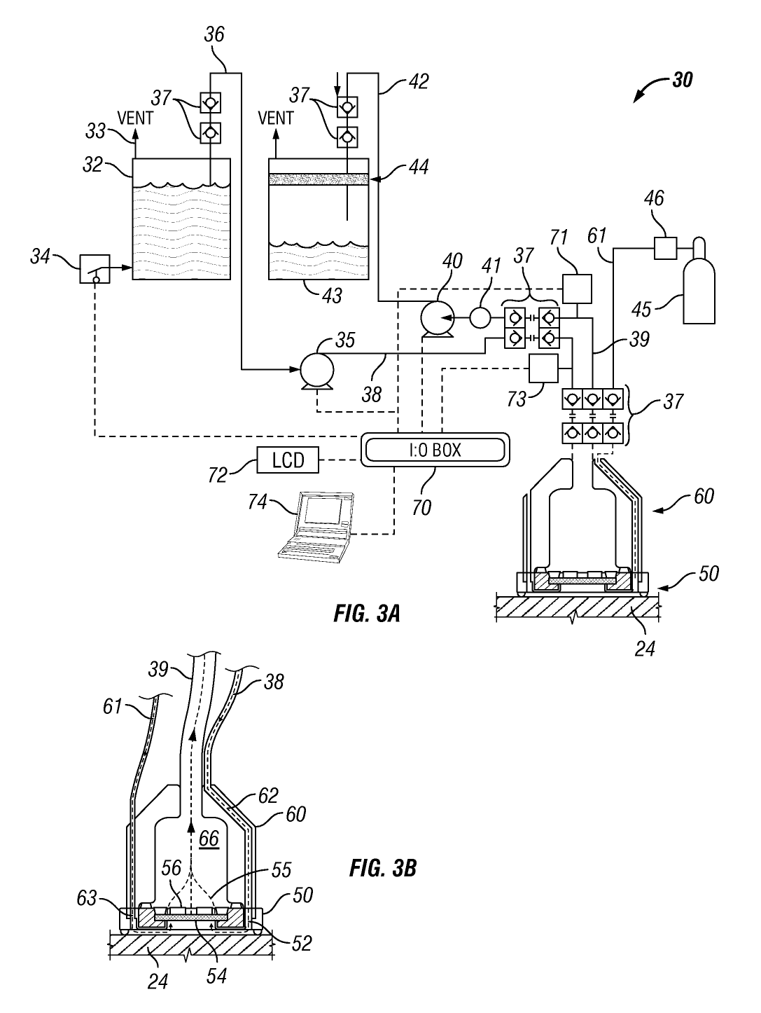

FIG. 3A is a flow diagram of an apparatus 30 for collecting samples. This apparatus is similar to apparatus 10 of FIGS. 1-2B, but provides more detail and includes the capability of using both a pressurized fluid source and a vacuum pressure drain. First the apparatus 30 has a rinse solution subsystem, including a rinse solution reservoir 32 with a gas vent 33 and low level sensor or switch 34. Rinse solution is drawn from the reservoir 32 using a pump 35 that is in fluid communication with the reservoir through conduit 36, preferably including quick disconnects 37 for ease of use particularly in replacing the rinse solution. The outlet of the pump 35 sends the rinse solution through tubing 38 to the sampling device 60, discussed later.

The apparatus 30 also has a fluid drain subsystem, including conduit 39 that leads from the sampling device 60 to a suction pump 40, where the conduit preferably includes a flow meter 41. The outlet of the suction pump 40 sends fluid through a conduit 42 to a waste reservoir 43 where the fluid is collected, typically for batch-wise disposal. As fluid accumulates in the reservoir 43, a gas permeable membrane 44 is used to vent gas as it is displaced from the reservoir.

Further, the apparatus 30 includes a pressurized fluid subsystem that has a pressurized gas tank 45, such as a pressurized carbon dioxide cylinder, and pressure regulator 46 that are in fluid communication with the sampling device 60 through a conduit 61.

Referring briefly to FIG. 3B, the sampling device 60 is shown in greater detail with fluid connections with the fluid drain conduit 39, the fluid supply conduit 61, and the rinse solution conduit 38 (albeit in reversed order). Thus, the sampling device 60 operates in substantially the same manner as the sample device 12 of FIG. 1. Specifically, the optional rinse solution may be sprayed or otherwise released onto a surface to be sampled 24 by fluid communication from the conduit 38 through a passageway 62 of the sampling device 60 and through a channel 52 in the filter cartridge 50. The fluid supply conduit 61 is in fluid communication with a passageway through the sampling device 60, which is in fluid communication with a channel 63 in the filter cartridge 50. Finally, the fluid drain conduit 39 allows the fluid and any rinse solution to pass through the filter element 54 and macroporous support 55, any additional passageway 56 to exit the filter cartridge, a passageway 66 through the sampling device 60 on its way to the waste reservoir. The filter element 54 is responsible for trapping any biological or chemical material that is the target of analysis.

Referring back to FIG. 3A, the apparatus 30 may further include a control subsystem that allows the apparatus to be closely monitored and/or automated. An electronic input/output box 70 in electronic communication with the waste reservoir low level sensor 34, rinse solution pump 35, suction pump 40, and pressure transducers 71, 73. A liquid crystal display (LCD) 72 is incorporated to provide real-time information about the status of the equipment or the sample being collected. A computer 74 is in electronic communication with I/O box 70 to receive data signals and to send control signals. While the computer 74 could be replaced with an analog controller or other type of controller, a digital computer is preferred. However, the computer does not need to take the form of a desktop model as pictured. Furthermore, it should be recognized that the computer may in fact be any number of computers, as would be the case with distributed controllers that provide local control of certain functions. Regardless of the details of the computer 74, control logic for executing a sample collection procedure may be provided as software loaded into a memory device forming part of the computer. The system will preferably also include one or more user interface, such as a visual monitor, mouse or trackball, keyboard, and speakers.

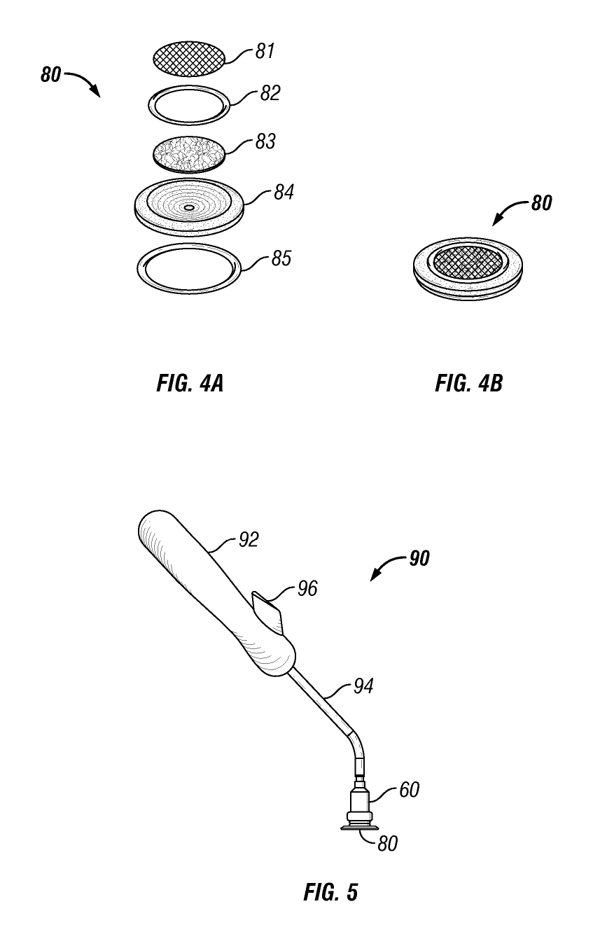

FIG. 4A is an exploded view of a disposable filter cartridge 80. The filter cartridge 80 includes (in order from top to bottom) a filter support 81, cartridge seal 82, filter element 83, porous filter holder 84, and sample surface seal 85. These components can, for example, be assembly by frictional engagement, adhesive bonding, or combinations thereof, to produce a unitary filter cartridge 80 as shown on the right. It should be recognized that it is not a requirement that the assembly be permanent. In fact, it may be preferred to be able to remove the filter element 83 from the rest of the cartridge components to facilitate analysis of the sample collected thereon. While the filter cartridge is preferably disposable, it may be reused after sterilization. Various factors may favor disposal, given the small size of the cartridge.

FIG. 4B shows the disposable filter cartridge 80 after assembly.

FIG. 5 is a perspective view of a self-contained sample collection apparatus 90 in the form of a wand. The disposable filter cartridge 80 is replaceably secured to the distal end of the sampling device 60. A water reservoir (fluid drain) and any optional fluid source or rinse solution reservoirs are housed in the handle 92 and conduits providing fluid communication between the reservoirs and the sampling device 60 extend through the tubular neck 94. Preferably, the handle 92 will include a button 96 that serves as a trigger to release and/or secure individual filter cartridges 80. In this manner, the filter cartridges do not have to be manipulated by hand. Various mechanical or electrical triggering devices will be apparent to one have ordinary skill in mechanical design.

### EXAMPLE 1

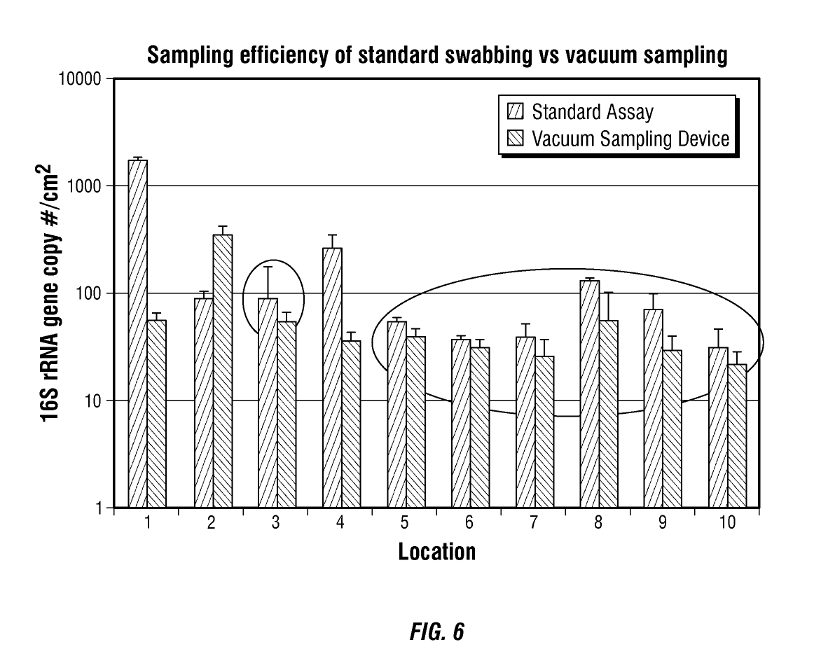

The sampling device that was used to generate FIG. 6 is the same as the wand from FIG. 5. In addition, the wand plugged into a base unit that provided vacuum assistance in the extraction of the samples from the surface being tested. The filter cartridge having the extracted samples where then stored in aseptic containers and used later for detection of the RNA.

FIG. 6 is a bar chart showing the relative amounts of RNA detected from samples collected by a standard wet swab technique and using the vacuum sampling device described in Example 1 at ten locations on a surface. The results produced by vacuum sampling compared well with results produced using the wet swab technique.

The terms “comprising,” “including,” and “having,” as used in the claims and specification herein, shall be considered as indicating an open group that may include other elements not specified. The term “consisting essentially of,” as used in the claims and specification herein, shall be considered as indicating a partially open group that may include other elements not specified, so long as those other elements do not materially alter the basic and novel characteristics of the claimed invention. The terms “a,” “an,” and the singular forms of words shall be taken to include the plural form of the same words, such that the terms mean that one or more of something is provided. For example, the phrase “a solution comprising a phosphorus-containing compound” should be read to describe a solution having one or more phosphorus-containing compound. The term “one” or “single” shall be used to indicate that one and only one of something is intended. Similarly, other specific integer values, such as “two,” are used when a specific number of things is intended. The terms “preferably,” “preferred,” “prefer,” “optionally,” “may,” and similar terms are used to indicate that an item, condition or step being referred to is an optional (not required) feature of the invention.

It should be understood from the foregoing description that various modifications and changes may be made in the preferred embodiments of the present invention without departing from its true spirit. It is intended that this foregoing description is for purposes of illustration only and should not be construed in a limiting sense. Only the language of the following claims should limit the scope of this invention.

What is claimed is:

1. An apparatus for sampling microorganisms, comprising:
a hand-held sampling device having a proximal end adapted for coupling in fluid communication with a fluid drain, a distal end including a coupling, and a first fluid passageway extending from the proximal end to the distal end; and
a replaceable sample collection cartridge having a proximal end adapted for selectively sealingly securing to the sampling device by engagement with the coupling, a second fluid passageway providing fluid communication between the first fluid passageway and a distal end of the cartridge, and a microorganism filter element extending across the second passageway and disposed a spaced distance from the distal end;
wherein a sample collection manifold is defined between the microorganism filter elements and the surface to be sampled, wherein the sample collection manifold provides fluid communication between a fluid source, the surface to be sampled, and the microorganism filter element, and wherein there is a sufficient differential pressure between the fluid source and the fluid drain to pass fluid from the fluid source across the surface to be sampled and through the microorganism filter element to the fluid drain such that a sample of microorganisms is collected from the surface to be sampled onto the microorganism filter element.

2. The apparatus of claim 1, wherein the sample collection cartridge can be disengaged from the coupling after collecting the sample and a subsequent sample collection cartridge can be engaged with the coupling for collection of a subsequent sample that is free of cross-contamination from any previous sample.

3. The apparatus of claim 1, further comprising a rinse reagent supply channel extending through the sample collection cartridge and terminating at the recessed face.

4. The apparatus of claim 1, wherein the fluid source is a gas selected from an inert gas, air, carbon dioxide, and combinations thereof.

5. The apparatus of claim 1, wherein the coupling is selected from a latch-in-place mechanism, a friction fitting, snap-on elements, and combinations thereof.

6. The apparatus of claim 1, wherein the sample collection cartridge is an aseptic disposable single-use cartridge stored in an aseptic container prior to coupling with the sampling device, and wherein the microorganism filter element does not come into contact with any other surface or fluid that has been cross contaminated.

7. The apparatus of claim 1, wherein the sample collection cartridge is aseptically dispensable from the sampling head into a container of recovery solution.

8. The apparatus of claim 1, wherein the depth of the recessed face provides turbulent fluid communication with a known reagent source.

9. The apparatus of claim 1, wherein the rinse reagent supply channel provides fluid communication with the sample collection manifold adjacent the perimeter of the manifold for delivering a rinse reagent across the surface to the sampled to the microorganism filter element.

10. The apparatus of claim 1, wherein the recessed surface is bounded by a sealing member that forms a temporary seal against the surface to be sampled.

11. The apparatus of claim 1, wherein the latch-in-place mechanism includes a quick release trigger for removing the sample collection cartridge from the sampling device.

12. The apparatus of claim 1, wherein the sample collection cartridge is operationally independent of the orientation of the surface to be sampled

13. The apparatus in claim 1, where the sample collection cartridge comes in various sizes to accommodate access to crevices.

14. The apparatus of claim 1, further comprising two or more sample collection cartridges with distal ends having different shapes for collecting samples from different surface geometries or locations.

15. The apparatus of claim 1, wherein the surface to be sampled is undamaged by the collection of the sample.

16. The apparatus of claim 1, wherein the fluid is aqueous.

17. The apparatus of claim 1, wherein the handheld device can sample surfaces, liquids and gases.

18. The apparatus of claim 1, wherein the microorganism filter elements is spaced less than 3 centimeters from the distal end of the sample collection cartridge.

19. The apparatus of claim 9, wherein the rinse reagent is selected from liquids, gases, and combinations thereof.
